(19) United States
(12) Patent Application Publication
(10) Pub. No.: US 2023/0312493 A1
(43) Pub. Date: Oct. 5, 2023

(54) DEUTERATED AND TRITIATED N-(4,5-DIMETHYLTHIAZOLO-2(3H)-YLIDEN)-2,2,3,3-TETRAMETHYLCYCLOPROPANE-1-CARBOXAMIDE DERIVATIVES AND THE USE THEREOF

(71) Applicant: HELMHOLTZ-ZENTRUM DRESDEN-ROSSENDORF E.V., Dresden (DE)

(72) Inventors: Rares-Petru MOLDOVAN, Dresden (DE); Rodrigo TEODORO, Dresden (DE); Magali TOUSSAINT, Dresden (DE); Daniel GÜNDEL, Dresden (DE); Winnie DEUTHER-CONRAD, Dresden (DE); Peter BRUST, Dresden (DE)

(73) Assignee: HELMHOLTZ-ZENTRUM DRESDEN-ROSSENDORF E.V., Dresden (DE)

(21) Appl. No.: 18/004,099

(22) PCT Filed: Jul. 9, 2021
(86) PCT No.: PCT/DE2021/100600

§ 371 Date: Jan. 3, 2023

(30) Foreign Application Priority Data
Jul. 10, 2020 (DE) 10 2020 118 255.4

(51) Int. Cl. C07D 277/18; A61K 51/04

(52) U.S. Cl. CPC C07D 277/18; A61K 51/0453

(57) ABSTRACT

Compounds of general formula I

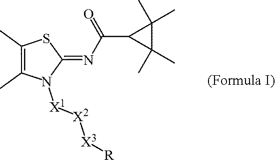

X¹ is a CD₂ group or a CT₂ group; X² is oxygen or a group (CZ¹Z²)ₙ, wherein Z¹ and Z² on each occurrence independently each are hydrogen, deuterium, or tritium and n is an integer from 1 to 12; X³ is a CD₂ group or a CT₂ group; and R is selected from the group consisting of hydroxy, -NO₂, halogen, a diazonium ion, a diazonium salt, a trialkylammonium ion, a trialkylammonium salt, a dialkoxyarene, a sulphoxide, a boronic acid, a boronic acid ester, an organotin compound, an iodonium ion, an iodonium salt, an iodonium ylide, and a sulphonate.

### FIELD

The invention relates to deuterated and/or tritiated N-(4,5-dimethylthiazole-2(3H)-ylidene)-2,2,3,3-tetramethylcyclopropane-1-carboxamide derivatives and the use thereof for the preparation of radio-fluorinated compounds. In particular, it relates to precursors for the preparation of radio-fluorinated deuterated and tritiated N-(4,5-dimethylthiazole-2(3H)-ylidene)-2,2,3,3-tetramethylcyclopropane-1-carboxamide derivatives, the use of the precursors for this purpose, and radio-fluorinated deuterated and tritiated N-(4,5-dimethylthiazole-2(3H)-ylidene)-2,2,3,3-tetramethylcyclopropane-1-carboxamide derivatives.

### BACKGROUND

From WO 2009/067613 A1 compounds of general formula S-1 are known

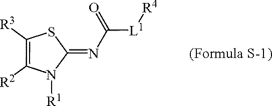

which are described as ligands for the cannabinoid receptor 2 (CNR₂ or CB₂). The compounds of general formula S-1 are said to be suitable as therapeutic agents for the treatment of diseases associated with CBR₂. The cannabinoid receptor 2 is a component of the endocannabinoid system and is predominantly present in the immune system. The receptor is involved in the regulation of processes in the body, in particular in the metabolism and immune functions. It mediates perception of pain and influences neuron activity. Affecting the cannabinoid receptor 2 can cause relief from pain, in particular chronic pain. Moreover, the receptor mediates the effect of cannabinoids such as (-)-Δ⁹-trans-tetrahydrocannabinol in the immune system.

In addition, the compounds known from WO 2009/067613 A1 are said to be suitable for the treatment of inflammatory diseases, immune diseases, neurological disorders, cancers of the immune system, respiratory tract diseases, and cardiovascular diseases.

Furthermore, the radio-labeled N-(4,5-dimethylthiazole-2(3H)-ylidene)-2,2,3,3-tetramethylcyclopropane-1-carboxamide derivatives shown below are known. Compound [¹¹C]A836339 was published by Horti et al. (Bioorg. Med. Chem. 2010, 14, 5202-5207). Based on the (4,5-dimethylthiazole-2(3H)-ylidene)-2,2,3,3-tetramethylcyclopropane-1-carboxamide backbone Moldovan et al. (J. Med. Chem. 2016, 59, 7840-7855 and J. Nuc. Med. 2015, 56, 53, 1048) have synthetized selective, fluorinated CBR₂ ligands and studied them in a mouse model for neuroinflammation (LPS). Among these compounds was [¹⁸F]JHU94620. Based on Moldovan et al. (J. Med. Chem. 2016, 59, 7840-7855) [¹⁸F]2f has been developed by Caille et al. (Mol. Pharmaceutics 2017, 11, 4064-4078).

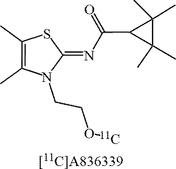

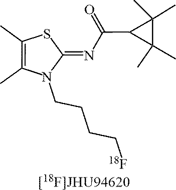

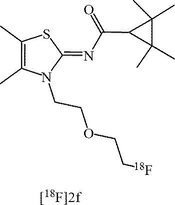

The ¹⁸F-labeled compound [¹⁸F]JHU94620 according to the prior art (Moldovan et al. J. Med. Chem. 2016, 59, 7840-7855) is prepared via radiosynthesis that starts with a bromine precursor. Here, the bromine precursor corresponds to [¹⁸F]JHU94620, except that there is a bromine atom as a leaving group instead of the [¹⁸F] atom. However, [¹⁸F]JHU94620 has low metabolic stability.

For a medical application of compound [¹⁸F]JHU94620 and other N-(4,5-dimethylthiazole-2(3H)-ylidene)-2,2,3,3-tetramethylcyclopropane-1-carboxamide derivatives said compounds should have higher metabolic stability.

### SUMMARY

The object of the invention is to eliminate the drawbacks according to the prior art. In particular, N-(4,5-dimethylthiazole-2(3H)-ylidene)-2,2,3,3-tetramethylcyclopropane-1-carboxamide derivatives are to be provided that not only have high affinity and selectivity over CBR₂, but also high metabolic stability and can be used as radiopharmaceuticals or precursors for the preparation of radiotracers, for example. Moreover, there is to be provided a method for the preparation of N-(4,5-dimethylthiazole-2(3H)-ylidene)-2,2,3,3-tetramethylcyclopropane-1-carboxamide derivatives which permits the preparation thereof with high radiochemical yields.

This object is solved by the features of claims 1, 8, 9, 11, 12, and 13. Suitable developments of the inventions result from the features of the dependent claims.

According to the invention a compound of general formula I is provided:

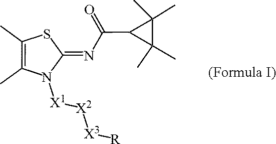

wherein
X¹ is a CD₂ group or a CT₂ group;
X² is oxygen or a group (CZ¹Z²)ₙ, wherein Z¹ and Z² on each occurrence independently each are hydrogen, deuterium or tritium and n is an integer from 1 to 12;
X³ is a CD₂ group or a CT₂ group; and
R is selected from the group consisting of hydroxy, -NO₂, halogen, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate.

A compound of general formula I may be its (E) isomer, its (Z) isomer or a mixture of the (E) and (Z) isomer.

A compound of general formula I may be provided, wherein
X¹ is a CD₂ group or a CT₂ group;
X² is selected from the group consisting of (CH₂)ₙ, (CD₂)ₙ, (CT₂)ₙ and O, wherein n is an integer from 1 to 12;
X³ is a CD₂ group or a CT₂ group; and
R is selected from the group consisting of hydroxy, -NO₂, halogen, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate.

If at least one of residues X¹, X², and X³ contains a deuterium atom, then it may be provided that residues X¹, X², and X³ contain no tritium atom. In this case, the compound of general formula I is either a deuterated compound or a tritiated compound, but no compound containing deuterium and tritium apart from their natural occurrence. Preferably, the compounds of general formula I according to the invention are deuterated compounds. The deuterated compounds preferably are no tritiated compounds. In the following, the term “deuterated compound” designates a compound containing deuterium atoms, but no tritium atoms apart from their natural occurrence. On the other hand, the term “tritiated compound” designates a compound containing tritium atoms, but no deuterium atoms apart from their natural occurrence.

There may be provided a compound of general formula I, wherein
X¹ is a CD₂ group;
X² is oxygen or a group (CZ¹Z²)ₙ, wherein Z¹ and Z² on each occurrence independently each are hydrogen or deuterium and n is an integer from 1 to 12;
X³ is a CD₂ group; and
R is selected from the group consisting of hydroxy, —NO₂, halogen, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate. Such a compound is a deuterated compound.

There may be provided a compound of general formula I, wherein
X¹ is a CD₂ group;
X² is selected from the group consisting of (CH₂)ₙ, (CD₂)ₙ and O, wherein n is an integer from 1 to 12;
X³ is a CD₂ group; and
R is selected from the group consisting of hydroxy, —NO₂, halogen, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate. Such a compound is a deuterated compound.

There may be provided a compound of general formula I, wherein
X¹ is a CD₂ group;
X² is (CH₂)ₙ or (CD₂)ₙ, wherein n is an integer from 1 to 12;
X³ is a CD₂ group; and
R is selected from the group consisting of hydroxy, —NO₂, halogen, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate. Such a compound is a deuterated compound.

According to the invention n is an integer from 1 to 12. It may be provided that n is 1, 2, 3, 4, 5, or 6. In one example n = 2. In this case, the N atom of the dimethylthiazole group bears an n-butyl group.

It may be provided that the compound of general formula I is a compound of general formula I-F or a compound of general formula I-P:

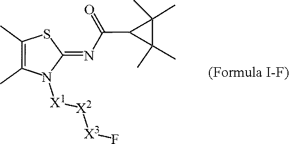

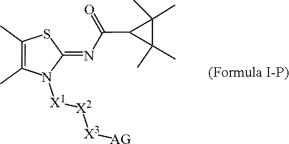

wherein X¹, X², and X³ have the meanings given in connection with formula I. AG designates a leaving group selected from the group consisting of hydroxy, —NO₂, chlorine, bromine, iodine, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate. A compound of general formula I-F may be its (E) isomer, its (Z) isomer or a mixture of the (E) and (Z) isomer. A compound of general formula I-P may be its (E) isomer, its (Z) isomer or a mixture of the (E) and (Z) isomer.

The compound of formula I-F is a compound of general formula I wherein R is fluorine. The compounds of formula I-F are deuterated and/or tritiated fluorinated compounds. It may be provided that the compound of general formula I is a compound of general formula [¹⁸F]I-F:

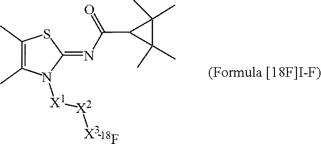

wherein X¹, X², and X³ have the meanings given in connection with formula I. The compound of formula [¹⁸F]I-F is a compound of general formula I wherein R is [¹⁸F]fluorine. The compounds of formula [¹⁸F]I-F are deuterated and/or tritiated radio-fluorinated compounds. A compound of general formula [¹⁸F]I-F may be its (E) isomer, its (Z) isomer or a mixture of the (E) and (Z) isomer.

The compounds according to the invention have high metabolic stability. This is a particular advantage of the compounds of general formula I-F and [¹⁸F]I-F because they are to be used in positron emission tomography (PET). The high metabolic stability strongly reduces the amount of metabolites in the brain (in vivo). Using compounds in PET imaging presumes that only small amounts of metabolites reach the brain. The compounds of general formula I according to the invention, in particular of general formula I-F, for example the compounds of general formula [¹⁸F]I-F, have high binding affinity and selectivity to CBR₂. Their binding affinity and selectivity correspond to that of the compounds having no deuteration or tritiation. Therefore, they can be used as highly affine and selective CBR₂ radiopharmaceuticals. The ¹⁸F-labeled radiotracers of general formula [¹⁸F]I-F according to the invention can be used for in vitro and in vivo studies of the expression and availability of CBR₂ in organisms by suitable detection methods such as positron emission tomography (PET). Said studies permit to gain more exact knowledges about the effect of reference compounds and thus, to develop further medicaments in addition to radiopharmaceuticals and to assess them in view of their potential efficacy.

The compound of general formula I-P is a compound of general formula I, wherein R is a leaving group AG. In particular, the compounds of general formula I-P can be used as precursors for the preparation of the compounds of general formula [¹⁸F]I-F.

It may be provided that AG is a sulphonate. The term “sulphonate” is meant to be an R^{S}—SO₂—O group. For example, R^{S} may be a branched or unbranched substituted or unsubstituted C₁-C₆ alkyl group, an aryl group, or an alkylaryl group. Preferably, R^{S} is CH₃—, CF₃—, or CH₃—C₆H₄—. For example, the sulphonate can be selected from the group consisting of a toluenesulfonic acid ester group, a methylsulfonic acid ester group, and a trifluoromethylsulfonic acid ester group. A toluenesulfonic acid ester group is meant to be a -OTs group, wherein Ts is tosyl. A methylsulfonic acid ester group is meant to be -OMs group, wherein Ms is mesyl. A trifluoromethylsulfonic acid ester group is meant to be CF₃—SO₂—O—. Preferably, AG is -OMs or -OTs. Particularly preferred AG is -OTs.

It may be provided that AG is an organotin compound. This may be tin organyl. For example, the organotin compound may be alkyl tin the alkyl group(s) of which can be substituted or unsubstituted, wherein one or more of the alkyl groups have optionally one or more hetero atoms, or aryl tin the aryl group(s) of which may be substituted or unsubstituted, wherein one or more of the aryl groups have optionally one or more hetero atoms. The hetero atoms may be for example O, N, S, or P.

It may be provided that AG is a halogen selected from the group consisting of chlorine, bromine, and iodine.

Preferred examples of compounds of general formula I are given in table 1. The compounds given in table 1 each may be their (E) isomer, their (Z) isomer or a mixture of the (E) and (Z) isomer.

TABLE 1

| Compound | Structure | Name |
|---|---|---|
| [¹⁸F]JHU94620-D4 | 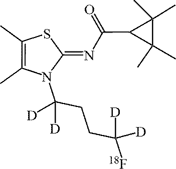 | N-(3-(4-([¹⁸F]fluoro)butyl- 1,1,4,4-d₄)-4,5-dimethylthiazole-2(3H)-ylidene)-2,2,3,3-tetramethylcyclopropane-1-carboxamide |
| JHU94620-D4 | 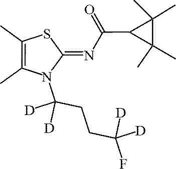 | N-(3-(4-fluorobutyl-1,1,4,4-d₄)-4,5-dimethylthiazole- 2(3H)-ylidene)-2,2,3,3-tetramethylcyclopropane-1-carboxamide |
| 3 | 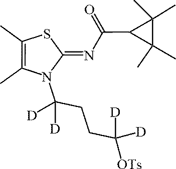 | 4-(4,5-dimethyl-2-((2,2,3,3-tetramethylcyclopropane-1-carbonyl)-imino)thiazole-3(2H)-yl)butyl-1,1,4,4-d₄-4- methylbenzenesulphonate |
| [¹⁸F]JHU94620-D8 | 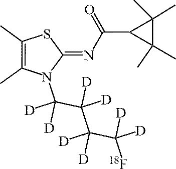 | N-(3-(4-([¹⁸F]fluoro)butyl- 1,1,2,2,3,3,4,4-ds)-4,5-dimethylthiazole-2(3H)-ylidene)-2,2,3,3-tetramethylcyclopropane-1-carboxamide |
| JHU94620-D8 | 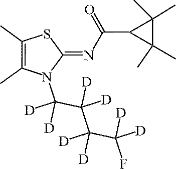 | N-(3-(4-fluorobutyl-1,1,2,2,3,3,4,4-ds)-4,5-dimethylthiazole-2(3H)-ylidene)-2,2,3,3-tetramethylcyclopropane-1-carboxamide |
| 4 | 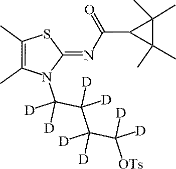 | 4-(4,5-dimethyl-2-((2,2,3,3-tetramethylcyclopropane-1-carbonyl)imino)thiazole-3(2H)-yl)butyl-1,1,2,2,3,3,4,4-ds-4-methylbenzenesulphonate |

Compounds [¹⁸F]JHU94620-D4 and [¹⁸F]JHU94620-D8 are compounds of general formula [¹⁸F]I-F. Compounds JHU94620-D4 and JHU94620-D8 are compounds of general formula I-F. Compounds 3 and 4 are compounds of general formula I-P. In particular, compound 3 can be used as a precursor for the preparation of [¹⁸F]JHU94620-D4. In particular, compound 4 can be used as a precursor for the preparation of [¹⁸F]JHU94620-D8.

It has been shown that the compounds of general formula I according to the invention, in particular of general formula I-F, represent ligands for the cannabinoid receptor 2. Therefore, they can be used for the diagnostics and therapy of inflammatory diseases, immune diseases, neurological disorders, cancers of the immune system, respiratory tract diseases, and cardiovascular diseases. Moreover, they can be used to relieve pain. Thus, the compounds of general formula I according to the invention can be used as a medicament, in particular as a medicament for inflammatory diseases, immune diseases, neurological disorders, cancers of the immune system, respiratory tract diseases, and cardiovascular diseases. Moreover, they can be used as a medicament for relieving pain. A pharmaceutically acceptable salt of a compound of general formula I may also be used as a medicament, in particular as a medicament for inflammatory diseases, immune diseases, neurological disorders, cancers of the immune system, respiratory tract diseases, and cardiovascular diseases or as a medicament for relieving pain. Inflammatory diseases comprise inflammations of nerve tissue, for example (which are also referred to as neuroinflammation).

According to the invention further provided is the use of a compound of general formula I-P

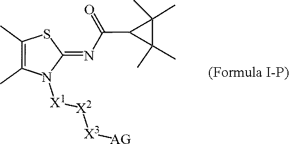

for the preparation of a compound of general formula [¹⁸F]I-F

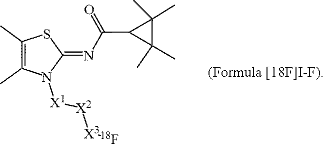

Here, the compound of general formula I-P corresponds to the compound of general formula [¹⁸F]I-F, except that the leaving group AG is replaced by [¹⁸F]fluorine. All the other substituents, i.e. X¹, X², and X³ including Z¹ and Z², are unchanged and are in the same position. Substituents X¹, X², X³, and AG in formulas I-P and [¹⁸F]I-F have the above-mentioned meanings.

The compounds of general formula I-P are particularly suitable precursors for the preparation of compounds of general formula [¹⁸F]I-F. For example, compound 3 can be used as a precursor for the preparation of [¹⁸F]JHU94620-D4.

According to the invention further provided is a method for the preparation of a compound of general formula I-F, e.g., a compound of general formula [¹⁸F]I-F. The method comprises the steps of:
(a) reacting a compound of formula 11

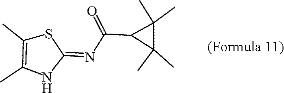

with a compound of general formula II

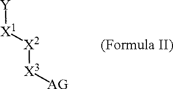

wherein X¹, X², X³, and AG have the meanings given in connection with general formula I-P and Y is a sulphonate, to a compound of general formula I-P; and
(b) reacting the compound of general formula I-P with a fluorinating agent to the compound of general formula I-F.

The compound of formula II may be its (E) isomer, its (Z) isomer or a mixture of the (E) and (Z) isomer.

In the compound of general formula II Y preferably is -O-Ts or -O-Ms, particularly preferred -O-Ts, wherein Ts designates a tosyl group and Ms a mesyl group. It may further be provided that AG also is a sulphonate. In this case, for example Y and AG each may have the same meaning. So, in a preferred embodiment both Y and AG each may be -O-Ts. One example of a compound of general formula II is butane-1,4-diyl-1,1,4,4-d₄-bis(4-methylbenzenesulphonate) (12). Another example of a compound of general formula II is butane-1,4-diyl-1,1,2,2,3,3,4,4-d₈-bis(4-methylbenzenesulphonate) (13).

The reaction of compound 11 with a compound of general formula II provided in step (a) of the method according to the invention may be carried out in an organic solvent. The organic solvent may be a non-protic, polar solvent such as acetonitrile (MeCN), dimethylformamide (DMF), N,N-dimethylacetamide (DMAA), N-methyl-2-pyrrolidone (NMP), dimethylsulfoxide (DMSO) or mixtures thereof. Preferably, dimethylformamide is used as the solvent. Preferably, the temperature is between 0 and 100° C., more preferably between 20 and 100° C., even more preferably between 40 and 80° C., especially preferred at 60° C. Preferably, the reaction is carried out at room temperature or a higher temperature. Preferably, the reaction is carried out for a period from 10 min to 2 hrs, more preferably from 30 min to 1.5 hrs and particularly preferred for 60 min. Preferably, the reaction is carried out at ambient pressure. Preferably, the reaction is carried out under agitating the reaction mixture, for example under stirring. Compound 11 and the compound of general formula II preferably are present in a molar ratio of 1:2 to 2:1, preferably of 1:1. The reaction may be carried out in the presence of a hydride, preferably an alkaline or alkaline-earth metal hydride. A preferred hydride is sodium hydride (NaH). The reaction may be carried out under a protective atmosphere, for example under a nitrogen or argon atmosphere. The concentration of compound 11, of the compound of general formula II and, if provided, of the hydride in the solvent, each should be at least 10% by weight relative to the solution.

Step (b) provides the reaction of the compound of general formula I-P with a fluorinating agent to the compound of general formula I-F. The choice of the fluorinating agent and the reaction conditions may depend on whether the compound of general formula I-F is a compound of general formula [¹⁸F]I-F or is not radio-fluorinated.

### Step (b): Reaction of a Compound of General Formula [¹⁸F]I-F

If the compound of general formula I-F is a compound of general formula [¹⁸F]I-F, then the fluorinating agent is [¹⁸F] fluoride anions. The [¹⁸F] fluoride anions may be present in a solution which in the following is referred to as [¹⁸F] fluoride-containing solution. In step (b) for the preparation of the [¹⁸F] fluoride-containing solution the [¹⁸F] fluoride anion can be prepared by means of known methods. For example, the [¹⁸F] fluoride anion is prepared in the cyclotron by radiating H₂¹⁸O, enriched to at least 97%, with protons of an energy of 9.6 MeV. The aqueous [¹⁸F] fluoride-containing solution obtained in this way may be fixed on an anion exchange cartridge (QMA) and eluted by means of an aqueous solution of a base, such as potassium carbonate, cesium carbonate, sodium hydride, or tetraalkylammonium hydrogen carbonate. Preferably, an aqueous solution of potassium carbonate is used as a base. Elution of the basic [¹⁸F] fluoride-containing solution takes place in a reaction vessel containing a phase transfer catalyst (PTC) such as crown ethers, quaternary ammonium salts or alkaline or alkaline-earth salts. As the PTC preferably a [2,2,2]-cryptand (Kryptofix® or K222), tetra-n-butyl-ammonium-phosphate, hydroxide, oxalate, toluene sulphonate, or optionally other crown ethers such as 18-crown-6 are used. The [¹⁸F] fluoride complex obtained in this way may be subjected to an azeotropic drying under vacuum. The organic solvent may be a non-protic, polar solvent such as acetonitrile (MeCN), dimethyl formamide (DMF), N,N-dimethyl acetamide (DMAA), N-methyl-2-pyrrolidone (NMP), dimethyl sulphoxide (DMSO), or mixtures thereof. Acetonitrile is preferably used as the solvent. Azeotropic drying is preferably carried out under thermal reaction control in the closed reaction vessel at an elevated temperature. The temperature is preferably between 50 and 60° C. Azeotropic drying may also be carried out with the support of microwaves. For that, microwaves with a power of 50 to 150 W, preferably 65 to 85 W and especially preferred 75 W may be used.

To carry out step (b) the azeotropically dried [¹⁸F]fluoride complex is preferably dissolved in an organic solvent. In this way the [¹⁸F]fluoride-containing solution is obtained. The organic solvent may be a non-protic, polar solvent such as acetonitrile (MeCN), dimethyl formamide (DMF), N,N-dimethyl acetamide (DMAA), N-methyl-2-pyrrolidone (NMP), dimethyl sulphoxide (DMSO), or mixtures thereof. Acetonitrile is preferably used as the solvent. Then, the precursor of general formula P-1 is added to this solution. Preferably, the precursor in advance is dissolved in an organic solvent. Said solvent preferably is the same solvent that is contained in the [¹⁸F]fluoride-containing solution as the solvent, i.e. acetonitrile. Then, the precursor dissolved in the solvent is added to the [¹⁸F]fluoride-containing solution. Step (b) of the method according to the invention is preferably carried out at an elevated temperature. The temperature is preferably between 80 and 110° C., especially preferred at 90° C. Preferably, step (b) is carried out for a period of 5 to 15 min and especially preferred 10 min. Step (b) of the method according to the invention is preferably carried out at ambient pressure. Step (b) is preferably carried out under agitation of the reaction mixture, for example under stirring.

### Step (b): Reaction of a Compound of General Formula I-F Containing No [¹⁸F]fluorine

If the compound of general formula I-F is not a compound of general formula [¹⁸F]I-F, then the fluorinating agent may be a compound that enables a nucleophilic fluorination of the compound of general formula I-P. For example, the fluorinating agent may be a tetraalkyl ammonium fluoride. The tetraalkyl ammonium fluoride may be selected from the group consisting of tetrabutyl ammonium fluoride (TBAF), tetraethyl ammonium fluoride (TEAF), and tetramethyl ammonium fluoride (TMAF).

Step (b) may be carried out in an organic solvent. The organic solvent may be a non-protic, polar solvent such as tetrahydrofuran (THF), acetonitrile (MeCN), dimethyl formamide (DMF), N,N-dimethyl acetamide (DMAA), N-methyl-2-pyrrolidone (NMP), dimethyl sulphoxide (DMSO), or mixtures thereof. Preferably, tetrahydrofuran is used as the solvent. The temperature is preferably between 0 and 100° C., more preferably between 20 and 80° C., even more preferred between 40 and 60° C., particularly preferred at 60° C. The reaction is preferably carried out at room temperature or at an elevated temperature. Preferably, the reaction is carried out for a period of 10 min to 2 hrs, more preferably of 30 min to 1.5 hrs and particularly preferred for 60 min. The reaction id preferably carried out at ambient pressure. The reaction is preferably carried out under agitation of the reaction mixture, for example under stirring. The compound of general formula I-P and the fluorinating agent are preferably present in a molar ratio from 1:3 to 2:1, preferably from 2:1. The reaction may be carried out under a protective atmosphere, for example under a nitrogen or argon atmosphere. The concentration of the compound of general formula I-P and of the fluorinating agent each should be at least 10% by weight relative to the solution.

Scheme 1 illustrates the preparation of a compound of general formula [¹⁸F]I-F according to the method according to the invention:

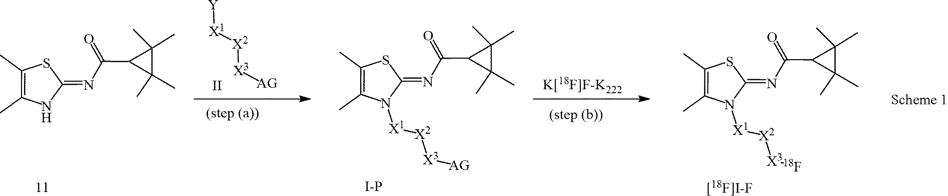

Scheme 2 illustrates the preparation of a compound of general formula I-F containing no [¹⁸F]fluorine according to the method according to the invention:

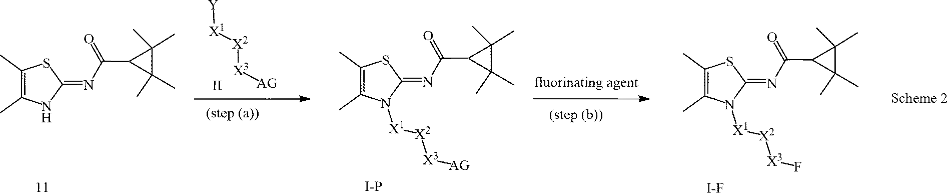

Step (a) permits the preparation of the compounds of general formula I-P. Thus, step (a) is a method for the preparation of a compound of general formula I-P. Scheme 3 illustrates the preparation of a compound of general formula I-P:

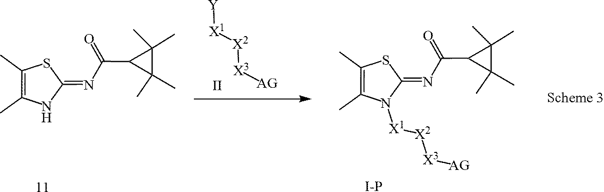

The procedure described in connection with step (a) may also be used for the preparation of a compound of general formula I-F, with the difference that a compound of general formula III

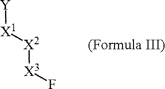

is used instead of a compound of general formula II. In the compound of general formula III X¹, X², and X³ have the meanings given in connection with general formula I-F. Scheme 4 illustrates the preparation of a compound of general formula I-F. Here, it may be provided that the compound III contains no [¹⁸F]fluorine.

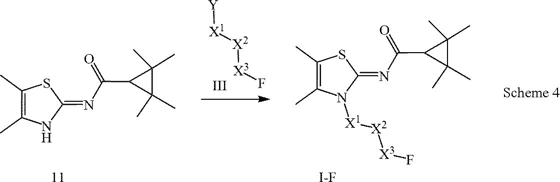

In particular, the invention enables the radiosynthesis of deuterated compounds of general formula [¹⁸F]I-F. In particular, the deuterated compounds of general formula [¹⁸F]I-F can be used as radiotracers for the nuclear-medical imaging of CBR₂ by means of positron emission tomography (PET). The deuterated compounds of general formula [¹⁸F]I-F have high affinity and selectivity to CBR₂.

It is known that hydrogen is a mixed element. In the preparation of compound [¹⁸F]JHU94620 known from the prior art therefore small amounts of deuterated and/or tritiated isotopologues may be obtained. However, the proportion of the deuterated and/or tritiated isotopologues is low, because it is determined by the natural frequency of deuterium and tritium. In the present invention each atom which is not explicitly named as specific isotope is a stable isotope. Unless otherwise stated a residue designated as hydrogen or H is meant to be a residue having hydrogen in its natural isotope ratio. Unless otherwise stated a residue designated as deuterium or D is meant to be a residue having deuterium in a frequency which is at least 3000 times higher than the natural frequency of deuterium. Assuming a natural frequency of deuterium of 0.015% a 3000 times higher frequency means an incorporation of deuterium of 45%. Unless otherwise stated a residue designated as tritium or T is meant to be a residue having tritium in a frequency that is at least 4.5*10¹⁶ times higher than the natural frequency of tritium. Assuming a natural frequency of tritium of 10⁻¹⁵ % a 4.5*10¹⁶ times higher frequency means an incorporation of tritium of 45%. The term “isotopologues” designates molecules that only differ in their isotope composition. They have the same chemical formula and the same bonding ratios between the atoms, but differ in at least one atom having a different number of neutrons.

The ratio between the frequency of an isotope in a compound and the natural frequency of the isotope is referred to as “isotope enrichment factor”. As described above, the isotope enrichment factor for each atom designated as deuterium should be at least 3000 (45% incorporation of deuterium in case of a residue designated as deuterium). The isotope enrichment factor can be at least 3500 (52.5% incorporation of deuterium), at least 4000 (60% incorporation of deuterium), at least 4500 (67.5% incorporation of deuterium), at least 5000 (75% incorporation of deuterium), at least 5500 (82.5% incorporation of deuterium), at least 6000 (90% incorporation of deuterium), at least 6333.3 (95% incorporation of deuterium), at least 6466.7 (97% incorporation of deuterium), at least 6600 (99% incorporation of deuterium) or at least 6633.3 (99.5% incorporation of deuterium). As described above, the isotope enrichment factor for each atom designated as tritium should be at least 4.5*10¹⁶ (45% incorporation of tritium in case of a residue designated as tritium). The isotope enrichment factor can be at least 5.25*10¹⁶ (52.5% incorporation of tritium), at least 6*10¹⁶ (60% incorporation of tritium), at least 6.75*10¹⁶ (67.5% incorporation of tritium), at least 7.5*10¹⁶ (75% incorporation of tritium), at least 8.25*10¹⁶ (82.5% incorporation of tritium), at least 9*10¹⁶ (90% incorporation of tritium), at least 9.5*10¹⁶ (95% incorporation of tritium), at least 9.7*10¹⁶ (97% incorporation of tritium), at least 9.9*10¹⁶ (99% incorporation of tritium) or at least 9.95*10¹⁶ (99.5% incorporation of tritium).

A compound which according to the invention has to have at least one deuterium atom or one tritium atom can be regarded as a group of isotopologues. The proportion of the isotopologues forming a compound may vary. A compound which according to the invention has to have at least one deuterium atom or one tritium atom contains small amounts of isotopologues having hydrogen atoms instead of one or more of the mentioned deuterium atoms or tritium atoms. The relative amount of said isotopologues should be less than 55% of the compound related to the compound. It may be provided that the relative amount of said isotopologues is less than 50%, less than 47.5%, less than 40%, less than 32.5%, less than 25%, less than 17.5%, less than 10%, less than 5%, less than 3%, less than 1% or less than 0.5%.

The term “alkyl”, unless stated otherwise, in particular relates to a saturated aliphatic hydrocarbon group having a branched or unbranched carbon chain of 1 to 12 carbon atoms, preferably 1 to 8 carbon atoms, and particularly preferred 1 to 6 carbon atoms. Examples of alkyl groups comprise but are not limited to methyl, ethyl, propyl, isopropyl, isobutyl, sec-butyl, tert-butyl, pentyl, n-hexyl, octyl, dodecyl, and the like. The alkyl group may optionally be substituted with one or more substituents, with each substituent independently being hydroxy, alkyl, alkoxy, halogen, haloalkyl, amino, monoalkylamino or dialkylamino, unless specifically stated otherwise.

The term “alkoxy”, unless stated otherwise, in particular relates to a group of formula -OR, wherein R is an alkyl group as defined herein. Examples of alkoxy components comprise but are not limited to methoxy, ethoxy, isopropoxy and the like. The alkoxy group may optionally be substituted with one or more substituents with each substituent independently being hydroxy, alkyl, alkoxy, halogen, haloalkyl, amino, monoalkylamino or dialkylamino, unless specifically stated otherwise.

The term “aryl” or “arene”, unless stated otherwise, in particular relates to a cyclic, aromatic hydrocarbon group consisting of a mono, bi or tricyclic aromatic ring system with 5 to 15 ring atoms, preferably 5 or 6 ring atoms. The aryl group may optionally be a substituted aryl group. Examples of aryl groups comprise but are not limited to phenyl, naphthyl, anthracenyl, naphthalenyl, phenanthryl, fluorenyl, indenyl, pentalenyl, azulenyl, oxydiphenyl, biphenyl, methylenediphenyl, aminodiphenyl, diphenyl sulfidyl, diphenyl sulfonyl, diphenyl isopropylideneyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl and the like, including partially hydrogenated derivatives thereof. A preferred example is phenyl. The term “substituted aryl group” in particular relates to an aryl group which is optionally substituted with one to four substituents, preferably one or two substituents selected from alkyl, cycloalkyl, heteroalkyl, hydroxyalkyl, halogen, nitro, cyano, hydroxy, alkoxy, amino, acylamino, monoalkylamino, dialkylamino, haloalkyl, haloalkoxy, urea, amido, alkane sulfonyl, -COR (wherein R is hydrogen, alkyl, phenyl, or phenylalkyl), -(CR′R″)ₙ-COOR (wherein n is an integer from 0 to 5, R′ and R″ independently are hydrogen or alkyl, and R is hydrogen, alkyl, cycloalkyl, cycloalkylalkyl, phenyl or phenylalkyl) or -(CR′R″)ₙ-CONR^{a′}R^{b′} (wherein n is an integer from 0 to 5, R′ and R″ independently are hydrogen or alkyl, and R^{a′} and R^{b′} independently are hydrogen, alkyl, cycloalkyl, cycloalkylalkyl, phenyl, or phenylalkyl). Examples of an arene comprise but are not limited to benzene, naphthalene, and anthracene.

The term “acyl”, unless stated otherwise, in particular relates to a group of formula -C(=O)R, wherein R is hydrogen or alkyl, as defined herein.

The term “halogen”, unless stated otherwise, relates to fluorine, chlorine, bromine, or iodine.

### BRIEF DESCRIPTION OF THE FIGURES

In the following the invention is explained in detail with the help of examples not intended to limit the invention with reference to the drawings. Here,

FIG. 1 shows an UV-HPLC chromatogram of the reference compound JHU94620-D4;

FIG. 2 shows a radio-HPLC chromatogram of compound [¹⁸F]JHU94620-D4 according to the invention;

FIG. 3 shows a radio-HPLC chromatogram of cerebral homogenate 30 p.i. of [¹⁸F]JHU94620; and

FIG. 4 shows a radio-HPLC chromatogram of cerebral homogenate 30 p.i. of [¹⁸F]JHU94620-D4.

### DETAILED DESCRIPTION

### Example 1 Synthesis of N-(3-(4-([¹⁸F]fluoro)butyl-1,1,4,4-d₄)-4,5-dimethylthiazole-2(3H)-ylidene)-2,2,3,3-tetramethylcyclopropane-1-carboxamide ([¹⁸ F]JHU94620-D4)

Scheme 5 shows the preparation of compound [¹⁸F]JHU94620-D4 according to the invention by reacting N-(4,5-dimethylthiazole-2(3H)-ylidene)-2,2,3,3-tetramethyl-cyclopropane-1-carboxamide (11) with butane-1,4-diyl-1,1,4,4-d₄-bis(4-methylbenzene sulphonate) (12) to the precursor (3) and the subsequent reaction of compound 3 to [¹⁸F]JHU94620-D4.

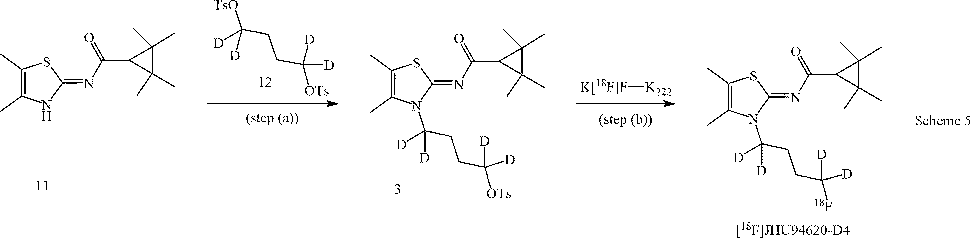

Compound 12 is a compound of general formula II, in which X¹ is CD₂, X² is (CH₂)₂, X³ is CD₂, Y is -OTs and AG is -OTs. K[¹⁸F]F-K₂₂₂ designates the [¹⁸F]F⁻/K₂₂₂/K⁺ complex the preparation of which is described below.

### Step (a): Synthesis of 4-(4,5-dimethyl-2-((2,2,3,3-tetramethylcyclopropane-1-carbonyl)-imino)thiazole-3(2H)-yl)butyl-1,1,4,4-d₄-4-methylbenzene sulphonate (3)

To a solution of compound 11 (1 eq, 0.6 mmol) and compound 12 (1.5 eq, 0.9 mmol) in 3 mL of DMF NaH (60%, 2 eq, 1.2 mmol) was added and the mixture was heated for 1 h to 60° C. under an argon atmosphere. Subsequently, the solvent was removed at the rotary evaporator. The residue was taken up in ethyl acetate (EA) (10 mL) and washed with an aqueous 5% NaHCO₃ solution (10 mL) and subsequently with a saturated NaCl solution (100 mL). Drying with MgSO₄ und removal of the solvents gave a yellow oil which was purified by means of column chromatography (silica gel, EA : petroleum ether (PE) of 1/20 to 1/4). Compound 3 was obtained as a white solid with a yield of 33%.

¹H-NMR (400 MHz, CDCl₃) δ 7.81 (d, J = 8.3 Hz, 2H), 7.36 (d, J = 8.0 Hz, 2H), 2.47 (s, 3H), 2.16 (s, 6H), 1.75 (m, 4H) 1.50 (s, 1H), 1.33 (s, 6H), 1.21 (s, 6H). HRMS (ESI+): m/z (%) = 483.2278, calc. 483.2284 for C₂₄H₃₁D₄FN₂O₄S₂⁺ [M+H]⁺.

### Step (b): Synthesis of N-(3-(4-([¹⁸F]fluoro)butyl-1,1,4,4-d₄)-4,5-dimethylthiazole-2(3H)-ylidene)-2,2,3,3-tetramethylcyclopropane-1-carboxamide ([¹⁸F]JHU94620-D4)

The aqueous [¹⁸F]fluoride (2 to 3 GBq) obtained after radiation was added to 1 mL of water, fixed on an anion exchange cartridge (QMA) and eluted with an aqueous K₂CO₃ solution (1.8 mg in 300 mL water) in a solution of 1 mL of MeCN and Kryptofix (K₂₂₂, 5.6 mg). Azeotropic drying of the complex was microwave assisted (power cycling, 75W, 50 - 60° C., argon stream) under vacuum. To the [¹⁸F]F⁻/K₂₂₂/K⁺ complex formed 2 mg of the precursor 3 (in 600 ml MeCN) were added. Subsequently, the reaction mixture was stirred at 90° C. for 10 min.

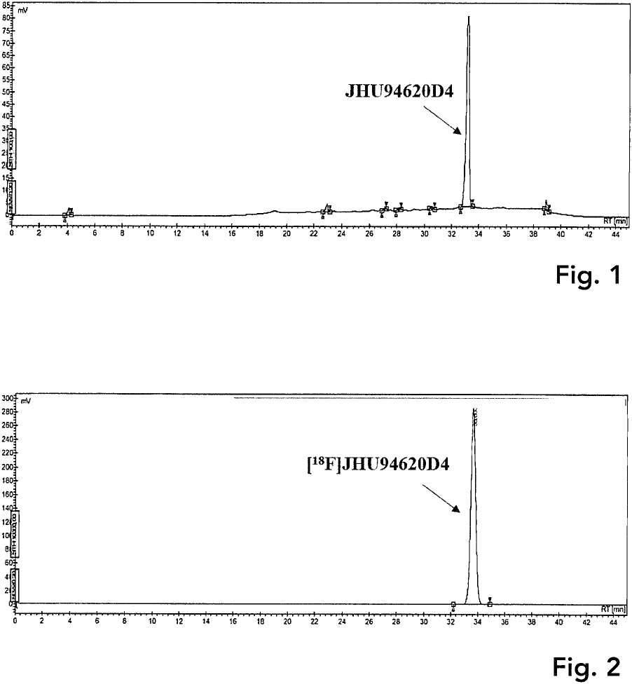

To determine the labeling yield an aliquot was taken and analyzed with radio-DC (49±4%, n=3) and radio-HPLC (48±5%, n=2). Purification and isolation of the radiotracer was carried out by means of semi-preparative RP-HPLC (column: ReproSil-Pur 120 C18-AQ, 250x20 mm, 5 mm; eluting agent: 65% MeCN/20 mM NH₄OAc_{aq.}; flow: 4.2 mL/min). The collected product fraction was diluted with water (20 mL), sorbed on an Sep-Pak^{®}-C18 Plus cartridge and eluted with ethanol (EtOH) (1.0 mL). Subsequently, the solvent was removed under heating in the argon stream and formulated in 0.9% saline (≤10% ethanol, v/v). The product [¹⁸F]JHU94620-D4 was isolated within a synthesis time of ca. 102 min and analyzed by radio-HPLC and radio-DC, whereby the identity of the product was confirmed by co-injection of the reference compound (see, FIGS. 1 and 2). The radiochemical purity was ≥ 99%. The radiochemical yield was about 20 to 25%. The molar activity was 200±20 GBq/µmol.

### Comparative Example 1 Synthesis of N-(3-(4-([¹⁸F]fluoro)butyl)-4,5-dimethylthiazole-2(3H)-ylidene)-2,2,3,3-tetramethylcyclopropane-1-carboxamide ([¹⁸F]JHU94620)

For comparison, [¹⁸F]JHU94620 was prepared using a bromine precursor, as described in Moldovan et al. J. Med. Chem. 2016, 59, 7840-7855. [¹⁸F]JHU94620 was obtained with a radiochemical yield of 16% and a molar activity of 170 GBq/µmol.

### Example 2 Synthesis of N-(3-(4-fluorobutyl-1,1,4,4-d₄)-4,5-dimethylthiazole-2(3H)-ylidene)-2,2,3,3-tetramethylcyclopropane-1-carboxamide (JHU94620-D4)

Scheme 6 shows the preparation of compound JHU94620-D4 according to the invention by reacting N-(4,5-dimethylthiazole-2(3H)-ylidene)-2,2,3,3-tetramethylcy-clopropane-1-carboxamide (11) with butane-1,4-diyl-1,1,4,4-d₄-bis(4-methylbenzene sulphonate) (12) to the precursor (3) and the subsequent reaction of compound 3 to JHU94620-D4.

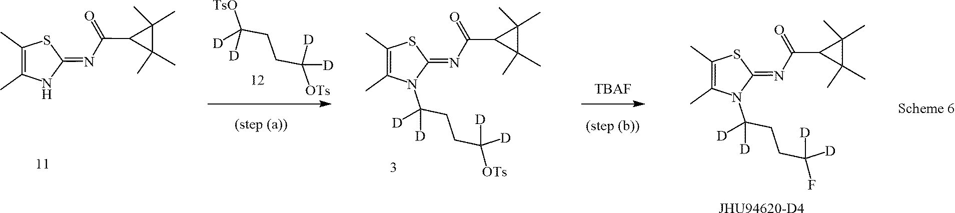

TBAF designates tetrabutyl ammonium fluoride.

### Step (a): Synthesis of 4-(4,5-dimethyl-2-((2,2,3,3-tetramethylcyclopropane-1-carbonyl)-imino)thiazole-3(2H)-yl)butyl-1,1,4,4-d₄-4-methylbenzene sulphonate (3)

Step (a) of the method shown in Scheme 6 was carried out as described in example 1, step (a).

### Step (b): Synthesis of N-(3-(4-fluorobutyl-1,1,4,4-d₄)-4,5-dimethylthiazole-2(3H)-ylidene)-2,2,3,3-tetramethylcyclopropane-1-carboxamide (JHU94620-D4)

To a solution of compound 3 (1 eq, 0.5 mmol) in 3 mL of THF TBAF (1 M in THF, 2 eq, 1 mmol) was added and the mixture was heated for 1 hr to 50° C. under an argon atmosphere. Subsequently, the solvent was removed at the rotary evaporator. The residue was taken up in ethyl acetate (EA) (10 mL) and washed with an aqueous 5% NaHCO₃ solution (10 mL) and subsequently with a saturated NaCl solution (100 mL). Drying with MgSO₄ und removal of the solvents gave a yellow oil which was purified by means of column chromatography (silica gel, EA : petroleum ether (PE) from 1/20 to 1/4). Compound JHU94620-D4 was obtained as a white solid with a yield of 72%.

¹H-NMR (300 MHz, CDCl₃) δ 2.20 (s, 3H), 2.18 (s, 3H), 1.55 (s, 1H), 1.36 (s, 6H), 1.24 (s, 6H). HRMS (ESI+): m/z (%) = 331.2152, calc. 331.2152 for C₁₇H₂₄D₄FN₂OS⁺ [M+H]⁺.

### Example 3 Synthesis of N-(3-(4-([¹⁸F]fluoro)butyl-1,1,2,2,3,3,4,4-d₈)-4,5-dimethylthiazole-2(3H)-ylidene)-2,2,3,3-tetramethylcyclopropane-1-carboxamide ([¹⁸F]JHU94620-D8)

Scheme 7 shows the preparation of compound [¹⁸F]JHU94620-D8 according to the invention by reacting N-(4,5-dimethylthiazole-2(3H)-ylidene)-2,2,3,3-tetramethyl-cyclopropane-1-carboxamide (11) with butane-1,4-diyl-1,1,2,2,3,3,4,4-ds-bis(4-methylbenzene sulphonate) (13) to the precursor (4) and the subsequent reaction of compound 4 to [¹⁸F]JHU94620-D8.

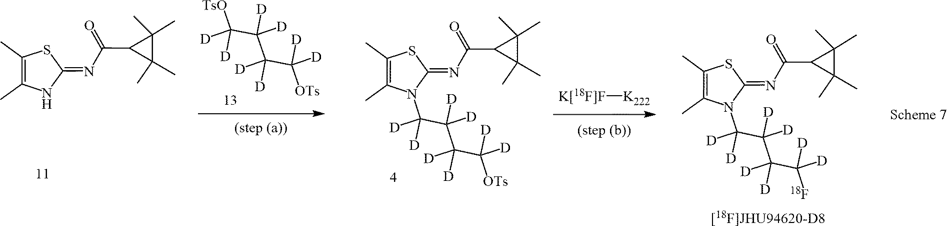

Compound 13 is a compound of general formula II, in which X¹ is CD₂, X² is (CD₂)₂, X³ is CD₂, Y is -OTs and AG is -OTs.

### Step (a): Synthesis of 4-(4,5-dimethyl-2-((2,2,3,3-tetramethylcyclopropane-1-carbonyl)-imino)thiazole-3(2H)-yl)butyl-1,1,2,2,3,3,4,4-d₈-4-methylbenzene sulphonate (4)

Compound 4 was prepared in the same way as in example 1, step (a), except that compound 13 was used instead of compound 12.

¹H-NMR (400 MHz, CDCl₃) δ 7.81 (d, J = 8.3 Hz, 2H), 7.36 (d, J = 8.0 Hz, 2H), 2.47 (s, 3H), 2.16 (s, 6H), 1.50 (s, 1H), 1.33 (s, 6H), 1.21 (s, 6H). HRMS (ESI+): m/z (%) = 487.2537, calc. 487.2535 for C₂₄H₂₇D₈N₂O₄S₂⁺ [M+H]⁺.

### Step (b): Synthesis of N-(3-(4-([¹⁸F]fluoro)butyl-1,1,2,2,3,3,4,4-d₈)-4,5-dimethylthiazole-2(3H)-ylidene)-2,2,3,3-tetramethylcyclopropane-1-carboxamide ([¹⁸F]JHU94620-D8)

[¹⁸F]JHU94620-D8 was prepared in the same way as in example 1, step (b), except that compound 4 was used instead of compound 3.

### Example 4 Synthesis of N-(3-(4-fluorobutyl-1,1,2,2,3,3,4,4-ds)-4,5-dimethylthiazole-2(3H)-ylidene)-2,2,3,3-tetramethylcyclopropane-1-carboxamide (JHU94620-D8)

Scheme 8 shows the preparation of compound JHU94620-D8 according to the invention by reacting N-(4,5-dimethylthiazole-2(3H)-ylidene)-2,2,3,3-tetramethylcyclopropane-1-carboxamide (11) with butane-1,4-diyl-1,1,2,2,3,3,4,4-ds-bis(4-methylbenzene sulphonate) (13) to the precursor (4) and the subsequent reaction of compound 4 to JHU94620-D8.

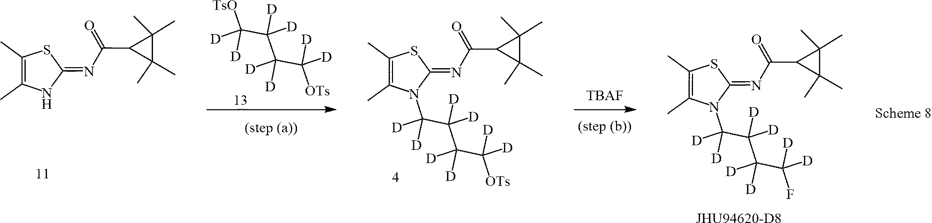

### Step (a): Synthesis of 4-(4,5-dimethyl-2-((2,2,3,3-tetramethylcyclopropane-1-carbonyl)-imino)thiazole-3(2H)-yl)butyl-1,1,2,2,3,3,4,4-d₈-4-methylbenzene sulphonate (4)

Step (a) of the method shown in scheme 8 was carried out as described in example 1, step (a).

### Step (b): Synthesis of N-(3-(4-fluorobutyl-1,1,2,2,3,3,4,4-d₈)-4,5-dimethylthiazole-2(3H)-ylidene)-2,2,3,3-tetramethylcyclopropane-1-carboxamide (JHU94620-D8)

JHU94620-D8 was prepared in the same way as in example 2, step (b), except that compound 4 was used instead of compound 3.

¹H-NMR (300 MHz, CDCl₃) δ 2.20 (s, 3H), 2.18 (s, 3H), 1.93 - 1.81 (m, 2H), 1.77 = 1.68 (m, 2H), 1.55 (s, 1H), 1.36 (s, 6H), 1.24 (s, 6H). HRMS (ESI+): m/z (%) = 335.2402, calc. 335.2403 for C₁₇H₂₀D₈FN₂OS⁺ [M+H]⁺.

### Example 5 Analysis of the Metabolic Stability of [¹⁸F]JHU94620-D4

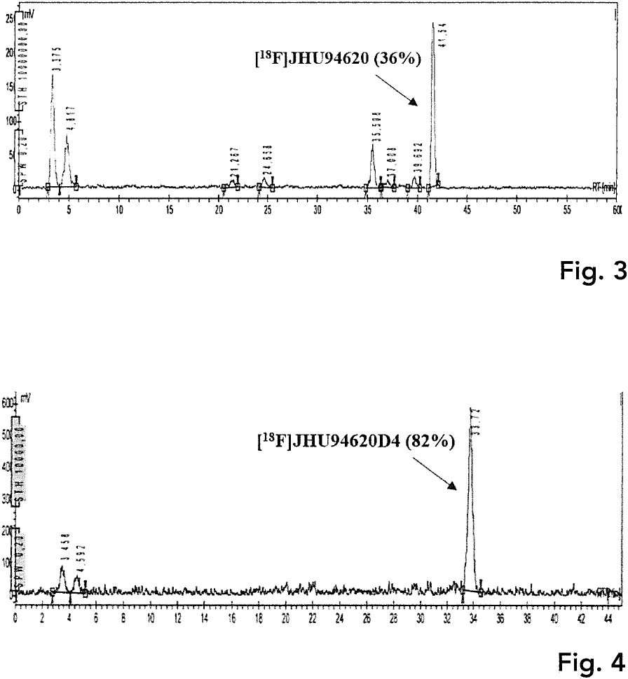

The metabolic stability of [¹⁸F]JHU94620-D4 was determined in accordance with the method described by Moldovan et al., J. Med. Chem. 2016, 59, 7840-7855. In the same way, the metabolic stability of JHU94620 and [¹⁸F]JHU94620 was determined. The radio-HPLC chromatograms shown in FIGS. 3 and 4 were determined using a ReproSil-Pur C18 AQ column (250×4.6 mm, 5 mm; eluting agent: 10-90-10% MeCN/20 mM NH₄OAc; flow: 1 ml/min). JHU94620 has the following formula:

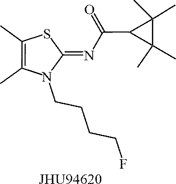

JHU94620 is the non-radio-fluorinated and non-deuterated analogue to [¹⁸F]JHU94620-D4 and [¹⁸F]JHU94620-D8.

It is seen in FIG. 4 that in the in vivo metabolite study in female CD-1 mice the percentage of intact radiotracer [¹⁸F]JHU94620-D4 in the brain was ~80% (radio-HPLC, extraction yield >97%) and in the plasma was ~15% (radio-HPLC, extraction yield >95%) 30 min after the injection. FIG. 3 shows that the percentage of intact radiotracer [¹⁸F]JHU94620 was only 38%. Thus, [¹⁸F]JHU94620-D4 has a substantially higher metabolic stability than [¹⁸F]JHU94620.

In the in vivo metabolite study in female CD-1 mice in case of JHU94620 the percentage of intact radiotracer in the brain was 36% (radio-HPLC, n=3, extraction yield 93%) and in the plasma was 7% (radio-HPLC, n=3, extraction yield 94%) of the total activity 30 min after the injection.

The metabolic stability of [¹⁸F]JHU94620-D8 is highly similar to that of [¹⁸F]JHU94620-D4.

### Example 6 Determination of the CBR₂ Equilibrium Dissociation Constant K_{D} of [¹⁸F]JHU94620-D8

The equilibrium dissociation constant K_{D} was determined by means of a homolog competition assay. For that, a constant amount of binding protein (homogenate of CHO cells that were stably transfected with human CBR₂ receptor; 2 Mio. cells/mL of the batch) was incubated with a constant amount of [¹⁸F]JHU94620-D8 (0.101 nM in the batch) and an increasing concentration of JHU94620 (0.01 nM-10 µM in the batch) for 60 minutes at room temperature in binding buffer (50 mM TRIS-HCl, pH 7.4, with 5 mM of MgCl₂, 1 mM EDTA, 1% BSA). Separation of receptor-bound and free radioligand was carried out by filtration over GF/B glass fiber filter incubated with 0.3% of polyethylene imine. Quantification of receptor-bound radioligand was carried out by measuring the filter-bound radioactivity in a gamma counter. Determination of the unspecific bonding of [¹⁸F]JHU94620-D8 was carried out by co-incubation with CP55940 (10 µM in the batch), determination of the adsorption of the radioligand at the glass fiber filter was carried out by incubation without binding protein. From the IC₅₀ value (2.82 nM) obtained in this experiment the K_{D} value of compound [¹⁸F]JHU94620-D8 over human CBR₂ receptors of the radioligands can be calculated via the Cheng-Prusoff equation simplified corresponding to the experimental approach (K_{D} = IC₅₀ (M) - radioligand (M)): K_{D} = 2.72 nM. CP55940 is a synthetic cannabinoid.

### Example 7 Determination of the CBR₂ Equilibrium Dissociation Constant Kᵢ of JHU94620

Using the Cheng-Prusoff equation

$${K}_{i} = \frac{I {C}_{50}}{\left(1 + \frac{L}{{K}_{D}}\right)}$$

the CBR₂ equilibrium dissociation constant Kᵢ of JHU94620 was determined. Here, L designates the concentration of the free ligand. Compound JHU94620 had a CBR₂ affinity of Kᵢ = 0.4 nM (n = 3). Replacing hydrogen atoms by deuterium ions shall not have any influence on the Kᵢ value. This also applies to the replacement of hydrogen atoms by tritium ions.

1-15. (canceled)

16. A compound of general formula I 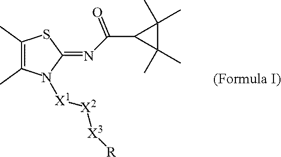
wherein
X¹ is a CD₂ group or a CT₂ group;
X² is oxygen or a group (CZ¹Z²)ₙ, wherein Z¹ and Z² on each occurrence independently each are hydrogen, deuterium, or tritium and n is an integer from 1 to 12;
X³ is a CD₂ group or a CT₂ group; and
R is selected from the group consisting of hydroxy, -NO₂, halogen, a diazonium ion, a diazonium salt, a trialkylammonium ion, a trialkylammonium salt, a dialkoxyarene, a sulphoxide, a boronic acid, a boronic acid ester, an organotin compound, an iodonium ion, an iodonium salt, an iodonium ylide, and a sulphonate.

17. The compound according to claim 16, wherein
X¹ is a CD₂ group or a CT₂ group;
X² is selected from the group consisting of (CH₂)ₙ, (CD₂)ₙ, (CT₂)ₙ, and O, wherein n is an integer from 1 to 12;
X³ is a CD₂ group or a CT₂ group; and
R is selected from the group consisting of hydroxy, -NO₂, halogen, a diazonium ion, a diazonium salt, a trialkylammonium ion, a trialkylammonium salt, a dialkoxyarene, a sulphoxide, a boronic acid, a boronic acid ester, an organotin compound, an iodonium ion, an iodonium salt, an iodonium ylide, and a sulphonate.

18. The compound according to claim 16, wherein
X¹ is a CD₂ group;
X² is selected from the group consisting of (CH₂)ₙ, (CD₂)ₙ, and O, wherein n is an integer from 1 to 12;
X³ is a CD₂ group; and
R is selected from the group consisting of hydroxy, -NO₂, halogen, a diazonium ion, a diazonium salt, a trialkylammonium ion, a trialkylammonium salt, a dialkoxyarene, a sulphoxide, a boronic acid, a boronic acid ester, an organotin compound, an iodonium ion, an iodonium salt, an iodonium ylide, and a sulphonate.

19. The compound according to claim 16, wherein n is 1, 2, 3, 4, 5, or 6.

20. The compound according to claim 16, wherein R is fluorine.

21. The compound according to claim 16, wherein R is [¹⁸F]fluorine.

22. The compound according to claim 16, wherein it is
N-(3-(4-([¹⁸F]fluoro)butyl-1,1,4,4-d₄)-4,5-dimethylthiazole-2(3H)-ylidene)-2,2,3,3-tetramethylcyclopropane-1-carboxamide;
N-(3-(4-fluorobutyl-1,1,4,4-d₄)-4,5-dimethylthiazole-2(3H)-ylidene)-2,2,3,3-tetramethylcyclopropane-1-carboxamide;
4-(4,5-dimethyl-2-((2,2,3,3-tetramethylcyclopropane-1-carbonyl)-imino)thiazole-3(2H)-yl)butyl-1,1,4,4-d₄-4-methylbenzene sulphonate;
N-(3-(4-([¹⁸F]fluoro)butyl-1,1,2,2,3,3,4,4-d₈)-4,5-dimethylthiazole-2(3H)-ylidene)-2,2,3,3-tetramethylcyclopropane-1-carboxamide;
N-(3-(4-fluorobutyl-1,1,2,2,3,3,4,4-ds)-4,5-dimethylthiazole-2(3H)-ylidene)-2,2,3,3-tetramethylcyclopropane-1-carboxamide; or
4-(4,5-dimethyl-2-((2,2,3,3-tetramethylcyclopropane-1-carbonyl)-imino)thiazole-3(2H)-yl)butyl-1,1,2,2,3,3,4,4-ds-4-methylbenzene sulphonate.

23. A compound according to claim 16 for use as a medicament.

24. A compound according to claim 16 for use as a medicament for the diagnostics and therapy of diseases in which a cannabinoid receptor 2 is involved.

25. The compound according to claim 23, wherein the medicament is a radiopharmaceutical for the nuclear-medical imaging of the cannabinoid receptor 2 by means of positron emission tomography (PET).

26. A medicament containing a compound according to claim 16 or a pharmaceutically acceptable salt thereof.

27. Use of a compound of general formula I-P 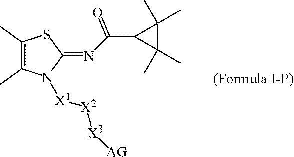
wherein
X¹ is a CD₂ group or a CT₂ group;
X² is oxygen or a group (CZ¹Z²)ₙ, wherein Z¹ and Z² on each occurrence independently each are hydrogen, deuterium, or tritium and n is an integer from 1 to 12;
X³ is a CD₂ group or a CT₂ group; and
AG is selected from the group consisting of hydroxy, -NO₂, chlorine, bromine, iodine, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate,
for the preparation of a compound of general formula [¹⁸F]I-F 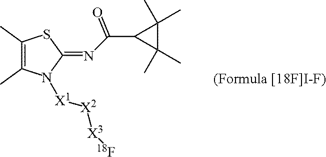
wherein X
¹, X², and X³ have the meanings given in connection with general formula I-P.

28. A method for the preparation of a compound of general formula I-F, 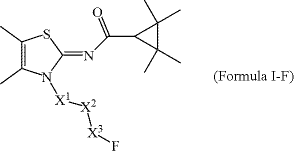
wherein
X¹ is a CD₂ group or a CT₂ group;
X² is oxygen or a group (CZ¹Z²)ₙ, wherein Z¹ and Z² on each occurrence independently each are hydrogen, deuterium, or tritium and n is an integer from 1 to 12; and
X³ is a CD₂ group or a CT₂ group;
wherein the method comprises the steps of:
(a) reacting a compound of formula 11 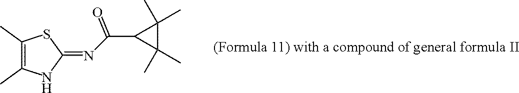 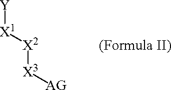
wherein
X¹, X², and X³ have the meanings given in connection with general formula I-P;
Y is a sulphonate; and
AG is selected from the group consisting of hydroxy, -NO₂, chlorine, bromine, iodine, a diazonium ion, a diazonium salt, a trialkylammonium ion, a trialkylammonium salt, a dialkoxyarene, a sulfoxide, a boronic acid, a boronic acid ester, an organotin compound, an iodonium ion, an iodonium salt, an iodonium ylide, and a sulphonate;
to a compound of general formula I-P 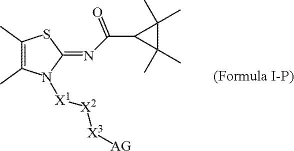
wherein X¹, X², X³, and AG have the meanings given in connection with general formula II; and
(b) reacting the compound of general formula I-P with a fluorinating agent to the compound of general formula I-F.

29. The method according to claim 28, wherein Y is -O-Ts or -O-Ms, wherein Ts designates a tosyl group and Ms a mesyl group.

30. The method according to claim 28, wherein Y is -O-Ts, wherein Ts designates a tosyl group.
